(19) United States
(12) Patent Application Publication
(10) Pub. No.: US 2016/0143766 A1
(43) Pub. Date: May 26, 2016

(54) Head Gear Assembly and Method

(71) Applicant: AccuMED Innovative Technologies, LLC, Buffalo, NY (US)

(72) Inventors: Amirtharajan Krishnasamy, West Seneca, NY (US); Michael DiMeo, Buffalo, NY (US)

(73) Assignee: ACCUMED INNOVATIVE TECHNOLOGIES, LLC, Buffalo, NY (US)

(21) Appl. No.: 14/552,680

(22) Filed: Nov. 25, 2014

(51) Int. Cl. A61F 5/37; B29C 65/04; A61M 16/06; A61F 5/01; A61F 5/56

(52) U.S. Cl. CPC A61F 5/3707; A61F 5/01; A61F 5/56; A61M 16/0683; B29C 65/04; A61M 2207/10

(57) ABSTRACT

A method for manufacturing a comfortable, wearable headgear device from individual components. At least two of the components are aligned at a first junction, and are welded together at the first junction using radio frequency welding. Additional components are welded to the partially assembled headgear device to comprise a final, comfortable, headgear device. The radio frequency welding results in junctions that do not chafe or become uncomfortable despite prolonged use.

### BACKGROUND

1. Field of the Invention

The present invention relates to medical headgear and, more particularly, to medical headgear fabrication methods.

2. Description of the Related Art

In medical device soft goods industries like orthopedic and sleep apnea products, laminated composites are widely used to build products which are utilized to hold medical devices or provide compression support to the users. Headgear and masks must be comfortable so that patients can wear the mask at night while they sleep. As a result, there is a continuous need in the art for headgear and masks that are comfortable, fit a wide range of patients, are easily manufactured, and are inexpensive.

The conventional/traditional laminated composite used for medical devices like headgear is typically a three layer or five layer material which consists of an open cell polyurethane foam, flame laminated with fabrics on either sides. This laminated composite is converted as usable soft goods medical products such as headgears which holds respiratory masks for sleep apnea. In some laminates, such as Breath-O-Prene®, a hook-engageable fabric is laminated to a polyurethane foam on one side, and fabric on the other. Silver-Tec® brand laminate is similar to a polyurethane laminate and contains silver to provide anti-microbial properties. Despite the perforations in these products, air permeation is limited. In some other cases an open, net-like fabric is provided, as in the Powerstretch-Rx® product available from Polartec and used on some Futuro® brand straps made by Biersdorf Inc. of Wilton, Conn., USA. This fabric is a non-hook-engageable knit spacer fabric with top and bottom fabric layers and yarns knit between the layers to space them apart. It is said to have moisture-releasing properties. Other brace straps, like the Futuro® Infinity precision-fit braces, advertise enhanced breathability and anti-microbial support. These laminates are foam laminates with a soft interior skin contact fabric laminated to a foam core, then laminated to an outer layer of hook-engageable loop material with holes on one side of the laminate that allow for some air and moisture transport.

These headgear devices are traditionally made using a combination of manufacturing methods like die cutting, sewing, and welding. Indeed, the traditional process involves various stages of operation to produce one complete finished headgear. Accordingly, there is a continued need for quick, efficient, and inexpensive methods of manufacture for headgear and other medical devices.

### BRIEF SUMMARY

The present disclosure is directed to methods for medical headgear fabrication. According to one aspect is a method for manufacturing a headgear device from at least two components, comprising the steps of: aligning, at a first junction, at least two components of the headgear device; welding, using radio frequency welding, the at least two components of the headgear device together at the first junction; and repeating, if necessary, at a second junction.

According to an embodiment, the method includes the steps of providing a source material for the at least two components of the headgear device; and generating the at least two components of the headgear device from the source material.

According to an embodiment, the step of generating the at least two components of the headgear device from the source material comprises radio frequency welding.

According to an embodiment, the step of generating the at least two components of the headgear device from the source material comprises die cutting and/or computer numerical controlled (CNC) cutting.

According to an embodiment, the step of generating the at least two components of the headgear device from the source material comprises compression molding.

According to an embodiment, a die is used to at least partially align the two components.

According to an embodiment, the method includes the steps of aligning, at a junction, at least two other components of the headgear device, and welding, using radio frequency welding, the at least two other components of the headgear device together at the third junction.

According to an embodiment, at least three of the at least two components are welded together at the first junction.

According to an embodiment, the source material is a soft, layered material, a foam layer, and/or an elastic.

According to an embodiment, each of the components of the headgear device comprises a unique component thickness, and the junction of the at least two components of the headgear device comprises a first thickness, such that after welding the first thickness is approximately equal to or less than at least one of the unique component thicknesses.

According to an aspect this invention describes a headgear device comprising at least two individual components welded together at a first junction by radio frequency welding.

According to an embodiment, the at least two individual components of the headgear device are welded together at a second junction by radio frequency welding.

According to an embodiment, at least one of the at least two individual components comprise a soft, layered material, a foam layer, and/or an elastic.

According to an aspect is a system for manufacturing a headgear device from at least two components, the system comprising at least two components of the headgear device, a die configured to align at least two of the components of the headgear device at a first junction, and a radio frequency welder configured to weld the at least two of the components of the headgear device at the first junction.

According to an aspect is a system for manufacturing a headgear device from at least two weldable components, the method including the steps of: (i) aligning, using a die, all of the at least two weldable components of the headgear device; and (ii) simultaneously welding, using radio frequency welding, the at least two components of the headgear device together at a plurality of junctions. The die can also seal together an edge of each of two or more of the at least two weldable components.

### BRIEF DESCRIPTION OF THE SEVERAL VIEWS OF THE DRAWING(S)

The present invention will be more fully understood and appreciated by reading the following Detailed Description in conjunction with the accompanying drawings, in which:

FIG. 1 is a graphical representation of headgear in accordance with an embodiment; and

FIG. 2 is a flow chart illustrating a method of radio frequency welding of medical device components in accordance with an embodiment.

### DETAILED DESCRIPTION

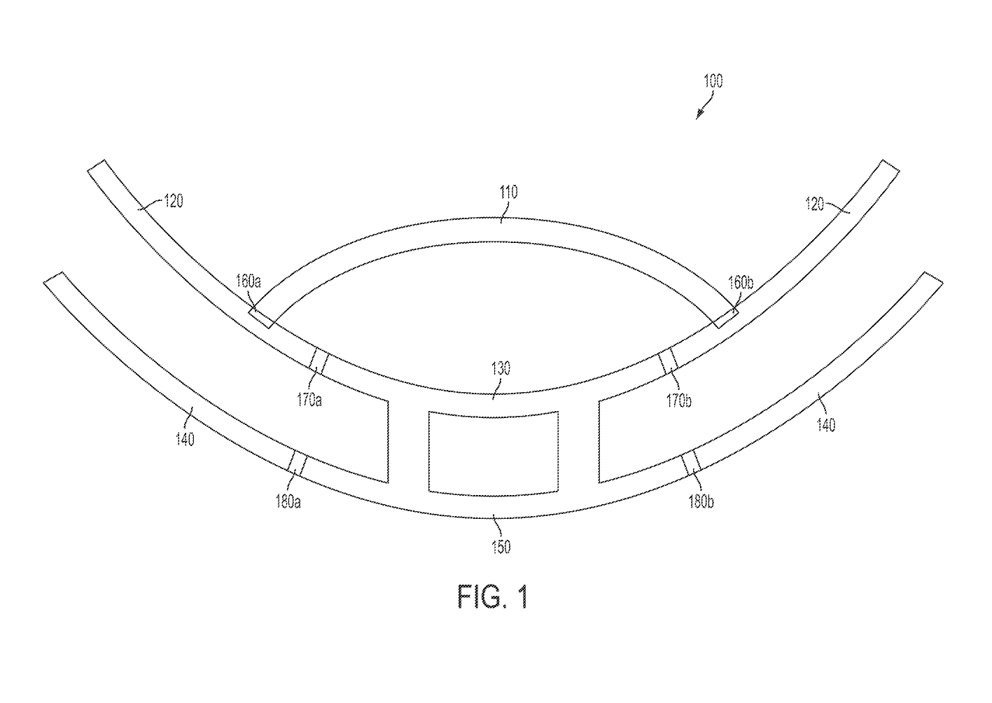

Referring now to the drawings, wherein like reference numerals refer to like parts throughout, there is seen in FIG. 1 a medical headgear device 100. Although particular embodiments of headgear are described or otherwise envisioned herein, one of skill in the art will recognize that the inventive methods can be utilized for a wide variety of medical devices, including headgear and many other devices which require comfortable fit.

Medical headgear device 100 can comprise, for example, two or more parts that must be permanently combined in a manner that provides comfort to the user. For example, the medical headgear device in FIG. 1 is comprises of a forehead strap 110, a top portion 120 with a second strap 130, and a bottom portion 140 with a chin strap 150. In order to function, the forehead strap, top portion, and bottom portion must be permanently combined in a manner that provides comfort to the user. Thus, there are junctions located at 160a and 160b, 170a and 170b, and at 180a and 180b.

Each of forehead strap 110, top portion 120, and bottom portion 140 can be composed of the same or different materials. For example, the components can be composed of soft, fabric that is comfortable to the skin. The fabric may also exhibit stretch, elasticity, and/or wicking properties. Examples include, but are not limited to a nylon-LYCRA® blend, and microfiber knit fabric. The primary benefit of the fabric is to provide minimal friction with the user's skin. It is preferable that the knit fabric is porous and allows airflow to reduce perspiration. The skin-facing fabric may be treated with a chemical that promotes wicking, i.e., transportation of moisture, through to the next layer of the assembly.

One or more of forehead strap 110, top portion 120, and bottom portion 140 can be composed of foam or a foam layer to provide cushioning. The foam or foam layer is preferably breathable, aiding in allowing moisture to move away from the skin toward the outside atmosphere by means of capillary attraction. The foam may be fabricated of a cellular, stretch-elastomer foam, including but not limited to polyurethane foam. The source material may also include one or more of an elastic, a film, non-woven fabric, a knitted fabric, a elastomer, a molded plastic, an extruded plastic, a woven fabric coated polymer or laminated with film, a synthetic or natural rubber, a spacer fabric, a molded hook, and a woven hook.

One or more of forehead strap 110, top portion 120, and bottom portion 140 can be composed of a rigid layer or rigid material to provide integrity to the structure without the need for an external support or structure (such as rigidizers or yokes that are typically applied to similar structures on the external surface). The material may be any plastic, composite, metal, or ceramic material having enough strength to provide integrity to the structure, but also allows for some flexibility. Examples of materials include, but are not limited to, thermoplastic and fiber reinforced thermoplastic materials. Examples of thermoplastic materials include, but are not limited to, polyamides. Examples of polyamides include, but are not limited to, nylon.

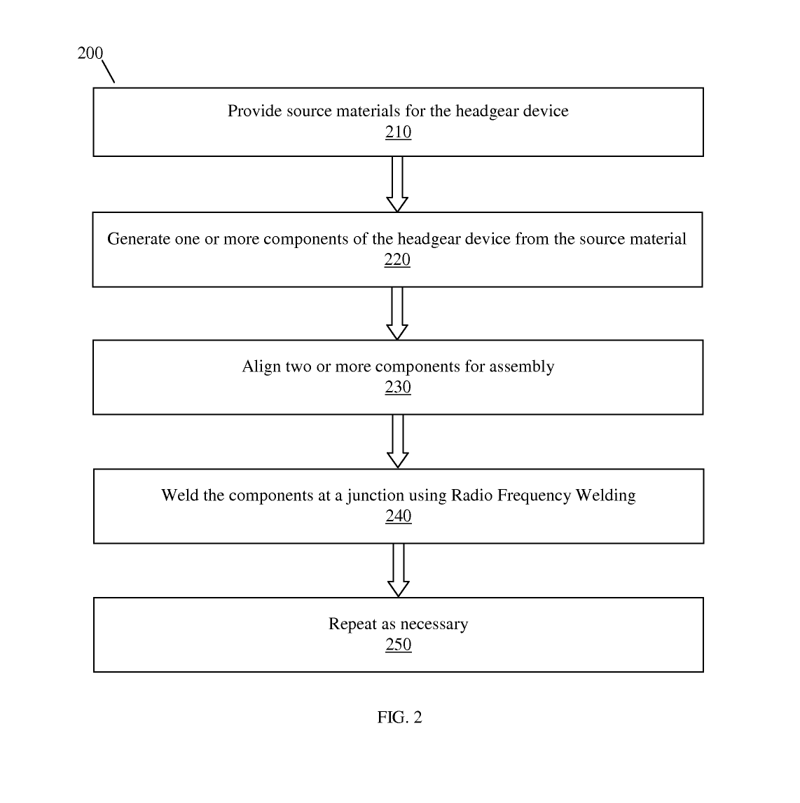

Referring to FIG. 2, a flow chart illustrating a method 200 for fabricating a medical device in accordance with an embodiment of the invention is disclosed. In step 210 of the method, a laminated source material is provided for the headgear device. The source material may be any material described or otherwise envisioned herein.

In step 220 of the method, the components are cut out of the source material using any of a variety of methods. Alternatively, the components are provided individually. For example, the unassembled components can be purchased from one or more suppliers. According to another embodiment, the source materials are generated using radio frequency welding, die cutting, computer numerical controlled (CNC) cutting, laser cutting, ultrasonic cutting, and/or compression molding.

In step 230 of the method, two or more of the individual components of the device to be assembled are aligned for assembly. For example, referring to the headgear depicted in FIG. 1, forehead strap 110 and top portion 120 can be aligned at junction 160a for welding. According to one embodiment, dies are used to direct the welding process, and forehead strap 110 and top portion 120 are properly aligned on a portion of a press in the die. The die may also weld together the aligned edges of two or more of the components of the device, thereby further facilitating the welding process.

In step 240 of the method, radio frequency welding is used to weld two components of the device, such as forehead strap 110 and top portion 120 (at junction 160a), together. Radio frequency welding uses electromagnetic energy to permanently bond the components together, which may or may not be composed of the same material. In other words, high frequency waves cause the molecules in the component materials to vibrate and gain energy, and the combination of heat and pressure causes the junction to weld in the shape of the die. Unlike other welding methods, the radio frequency welding combines the two components of the device, such as forehead strap 110 and top portion 120, at a junction while simultaneously providing a superior level of comfort to the user.

In step 250 of the method, radio frequency welding is used to weld two other components of the device together. For example, the welding method can be used to weld forehead strap 110 and top portion 120 at junction 160b. Step 250 can be repeated as necessary until all junctions of the device are welded or all junctions 160a, 160b, 170a and 170b, and 180a and 180b can be welded in one step after aligning the headgear components 110, 120, 130, 140 and 150 in position. Referring to the embodiment depicted in FIG. 1, junctions 160a, 160b, 170a and 170b, and 180a and 180b can all be welded.

There are numerous benefits gained from the use of radio frequency welding to manufacture the headgear device. For example, the material of two components is compressed at a junction resulting in a thickness of the welded area that is less than the sum of thickness of the first component and second component. As a result, multiple components can be assembled at one junction without increasing the total thickness of the overlapped area. Further, the geometry of the welded junction can be changed using radio frequency welding, and the junction can have customizable finished thicknesses. Further, either a stiff or a soft junction can be obtained, and because of the nature of the junction, it can be utilized as a hinge or a pivot without significantly weakening the weld. As yet another benefit, the pattern on the junction can be customized to any required pattern. The welding seals the edge of the material to produce sealed edge components, while allowing the sealing of the desired area while leaving other areas open.

In contrast, using traditional combination methods and welding methods, the materials can be softened, melted, and fused together, which can result in material failure under high shear forces.

Radio frequency welding is compatible with wide range of materials. For example, in accordance with an embodiment, a fabric/foam/fabric laminated composite like breathe-O-Prene can be utilized for manufacturing headgear. One or more of the components of the headgear can be made from materials including thermoplastic elastomers, molded thermoplastics, extruded thermoplastics, knitted fabrics, woven fabrics, spacer fabric, films, woven fabric coated polymer or laminated with film, non-woven fabrics, and compression molded (thermoformed or cold formed) material, among other materials. One or more of these materials can be combined with each other to form a finished product. Two or more layers of any these materials, including with varying thickness, are compatible with radio frequency welding.

In addition to assembling the components of the headgear, radio frequency welding can provide a number of other advantages to the finished product. For example, the wide range of material compatibility and flexibility of using radio frequency welding with preprocessed components enables the radio frequency welding to produce not only headgear, but a hybrid combination of the headgear, frame, and/or mask. In other words, the radio frequency welding can be used to create a combination of devices. Further, molded plastic or elastomeric parts can be used as a rigidiser to increase the rigidity or support at desired location to achieve required form fit or functions.

Although the present invention has been described in connection with a preferred embodiment, it should be understood that modifications, alterations, and additions can be made to the invention without departing from the scope of the invention as defined by the claims.

What is claimed is:

1. A method for manufacturing a headgear device from at least two components, the method comprising the steps of:
aligning, at a first junction, at least two components of the headgear device;
welding, using radio frequency welding, the at least two components of the headgear device together at the first junction; and
repeating, if necessary, at a second junction.

2. The method of claim 1, further comprising the steps of:
providing a source material for the at least two components of the headgear device; and
generating the at least two components of the headgear device from the source material.

3. The method of claim 2, wherein the step of generating the at least two components of the headgear device from the source material comprises radio frequency welding.

4. The method of claim 2, wherein the step of generating the at least two components of the headgear device from the source material comprises die cutting and/or computer numerical controlled (CNC) cutting.

5. The method of claim 2, wherein the step of generating the at least two components of the headgear device from the source material comprises ultrasonic cutting and/or laser cutting.

6. The method of claim 2, wherein the step of generating the at least two components of the headgear device from the source material comprises compression molding.

7. The method of claim 2, wherein said source material comprises one or more of a foam layer, an elastic, a spacer fabric, a film, non-woven fabric, a knitted fabric, a elastomer, a molded plastic, an extruded plastic, a woven fabric coated polymer or laminated with film, a synthetic or natural rubber, a molded hook, and a woven hook.

8. The method of claim 1, wherein a die is used to at least partially align the two components.

9. The method of claim 1, further comprising the steps of:
aligning, at a junction, at least two other components of the headgear device; and
welding, using radio frequency welding, the at least two other components of the headgear device together at a third junction.

10. The method of claim 1, wherein at least three of the at least two components are welded together at the first junction.

11. The method of claim 1, wherein each of the at least two components of the headgear device comprise a unique component thickness, and further wherein the junction of the at least two components of the headgear device comprises a first thickness, the first thickness being approximately equal to or less than at least one of the unique component thicknesses.

12. A headgear device comprising at least two individual components welded together at a first junction by radio frequency welding.

13. The headgear device of claim 12, wherein the at least two individual components of the headgear device are welded together at a second junction by radio frequency welding.

14. The headgear device of claim 12, wherein at least one of the at least two individual components comprise a soft, layered material.

15. The headgear device of claim 12, wherein a source material for at least one of said at least two individual components comprises one or more of a foam layer, an elastic, a spacer fabric, a film, non-woven fabric, a knitted fabric, a elastomer, a molded plastic, an extruded plastic, a woven fabric coated polymer or laminated with film, a synthetic or natural rubber, a molded hook, and a woven hook.

16. A system for manufacturing a headgear device from at least two components, the system comprising:
at least two components of the headgear device;
a die configured to align at least two of the components of the headgear device at a first junction; and
a radio frequency welder configured to weld the at least two of the components of the headgear device at the first junction.

17. A method for manufacturing a headgear device from at least two weldable components, the method comprising the steps of:
aligning, using a die, all of the at least two weldable components of the headgear device; and
simultaneously welding, using radio frequency welding, the at least two components of the headgear device together at a plurality of junctions.

18. The method of claim 17, wherein the die also seals together an edge of each of two or more of the at least two weldable components.
